(19) United States
(12) Patent Application Publication
(10) Pub. No.: US 2019/0021463 A1
(43) Pub. Date: Jan. 24, 2019

(54) IMPROVED CARRIER FOR MAINTAINING PERSONAL INTEGRITY

(71) Applicant: Atos Medical AB, Horby (SE)

(72) Inventors: Ida Oja, Horby (SE); Stina Millgard, Satila (SE); Birgitta Lyrdal, Bjarred (SE); Elin Persson, Malmo (SE); Pram Sax Pernille, Hvidovre (SE)

(21) Appl. No.: 16/070,221

(22) PCT Filed: Jan. 2, 2017
(86) PCT No.: PCT/SE2017/050002

§ 371 Date: Jul. 13, 2018

(30) Foreign Application Priority Data
Jan. 13, 2016 (SE) 1650035-7

(51) Int. Cl. A45C 7/00; A45C 13/02; A45C 11/00; A61M 16/04

(52) U.S. Cl. CPC A45C 7/0095; A45C 13/02; G09B 19/00; A61M 16/0465; A45C 11/00

(57) ABSTRACT

A carrier for carrying tracheostomy accessories may include a foldable carrier structure. The foldable carrier structure may include at least two compartments. Each compartment of the at least two compartments may be structured and arranged to carry a type of tracheostomy accessory. The carrier may also include an informative guide identifying the type of tracheostomy accessory each compartment is structured and arranged to carry. When the carrier structure is folded in a closed state the at least two compartments may be folded onto one another such that the at least two compartments are inaccessible.

### TECHNICAL FIELD

This application relates to an improved carrier for maintaining the personal integrity of a user of tracheostomy appliances.

### BACKGROUND

Having a diagnosis necessitating a tracheostomy is of course a personal matter that many users of tracheostomy appliances wishes to keep private. As with all issues of personal hygiene, such whishes should be respected.

A patient that has undergone thracheostomy has had a severe impact on his/her personal life. Once leaving hospital for self care at home, the patient suffers from anxiety and uncertainty with regard to how to use the different products—especially during night time.

Contemporary carriers for tracheostomy appliances are constructed as normal toiletry bags, leaving all appliances open to inspection by bystanders, even if the user only whishes to access a simple cleaning wipe, exposing all appliances including such used for night wear.

Contemporary carriers have tried to alleviate this by affixing external compartments or pockets for holding cleaning swipes etc, but this has lead to some confusion and/or annoyance among users, now having to open several pockets for performing simple tasks adding to the time it takes and to possible embarrassment felt by some users.

It is therefore a need for an improved carrier that maintains the personal integrity of the user while increasing the sense of security, and is convenient to use and also does not add to the time required for performing simple tasks.

### SUMMARY

One object of the present teachings herein is to solve, mitigate or at least reduce the drawbacks of the background art, which is achieved by the appended claims, and specifically by a carrier arranged to carry tracheostomy accessories, said carrier comprising: at least two compartments, each compartment being arranged to carry a type of the tracheostomy accessories; an informative guide guiding the user as to which compartment is carrying which accessory type; and said at least two compartments being foldable onto one another whereby said compartments are rendered inaccessible in a closed state.

One benefit is that a user may access any accessory without confusion and without unnecessarily exposing the other appliances to the surroundings and relief of anxiety and stress associated with self care at home after surgery.

As the carrier is compartmentalized and that the compartments are accessed and exposed in the order of most probable use, wherein the more advanced accessories, such as for sleeping, are accessed lastly—and assumingly in a more private sphere—the personal integrity of the user is preserved. As each compartment carries the accessories needed for each occasion, there is no need to open more than one compartment thereby both making the carrier more convenient and also more time efficient to use.

Other features and advantages of the disclosed embodiments will appear from the following detailed disclosure, from the attached dependent claims as well as from the drawings.

### BRIEF DESCRIPTION OF DRAWINGS

The invention will be described below with reference to the accompanying figures wherein:

FIG. 1 shows a side view of a carrier in a closed state according to an embodiment of the teachings herein;

FIG. 2 shows a front view and a rear view of a carrier in an opened state according to an embodiment of the teachings herein;

FIG. 3 shows a front view of a carrier in a closed informative state according to an embodiment of the teachings herein;

FIG. 4 shows a front view of a carrier in a partially opened state according to an embodiment of the teachings herein;

FIG. 5 shows a front view of a carrier having an opened compartment according to an embodiment of the teachings herein and also showing a close up of the opened compartment;

FIG. 6 shows a front view of a carrier having a removable compartment according to an embodiment of the teachings herein; and

FIG. 7 shows a front view of a carrier wherein each compartment has been marked with a symbol indicating a categorization according to an embodiment of the teachings herein.

### DETAILED DESCRIPTION

The invention will now be disclosed with reference to a few example embodiments. Even though the invention is only disclosed with reference to a few examples, it should be noted that many variations and also combinations of the exemplifying embodiments are possible within the scope of the invention.

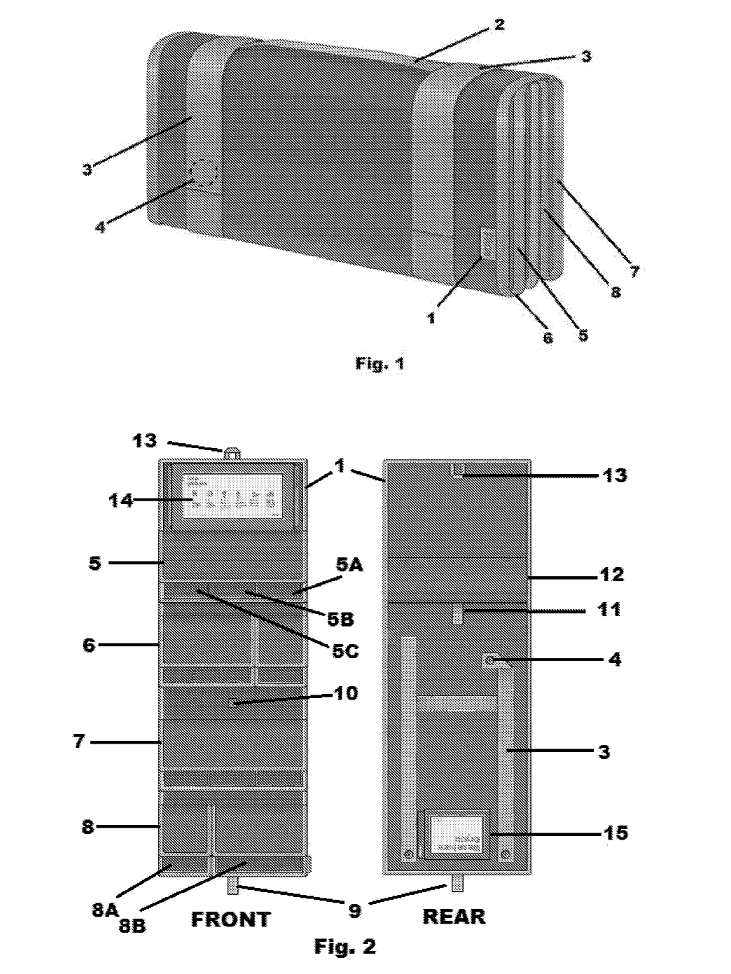

FIG. 1 shows a side view of a carrier 1 in a closed state according to an embodiment of the teachings herein. The carrier 1 comprises a carrying structure 1 which is foldable and when in a closed position, as in FIG. 1, the carrier is kept closed by one or more straps 3 that stretch at least partially around the carrier 1 and may be locked through locks 4, such as magnetic snaplocks, button locks, snap locks, hook and loop, such as through Velcro ^{TM} locks or other possible locking means. In one embodiment the locks 4 are magnetic as it provides for a fast release while still providing a sufficient locking force that is easily detachable. In one embodiment the locks 4 are of hook and loop type as it provides for a fast release while still providing a sufficient locking force that is easily detachable. The carrier 1 comprises two or more compartments, in this example the carrier 1 comprises four compartments 5, 6, 7 and 8. As seen in FIG. 1, the carrier 1 is folded so that the compartments 5, 6, 7 and 8 are engulfed and protected by the carrier 1. As the compartments 5, 6, 7 and 8 are folded in this manner they will exert a force both downwards (due to their weight), but also outwards thereby providing a locking force to the locking means 4.

In the closed state all compartments 5, 6, 7 and 8 are inaccessible, while providing for a small and compact overall size.

FIG. 2 shows a front view and a rear view of the carrier 1 in a fully opened state according to an embodiment of the teachings herein. In the front view of the fully opened state all four compartments 5, 6, 7 and 8 are accessible. It can be seen that each compartment 5, 6, 7 and 8 may comprise one or more pockets (exemplified only for the first compartment 5 and the fourth compartment which comprise three pockets 5A, 5B, 5C and two pockets 8A, 8B respectively. It should be noted that a compartment may comprise one, two or more pockets depending on the number and size of accessories to be carried in the compartment. The pockets may be covered by a flap as shown in FIG. 2. The pockets may also or alternatively be covered or closed by a zipper-like construction such as by a zipper or a zip lock.

In this example, the carrier has an informative guide 14 that may be shaped as a pocket for carrying manuals, recipes, medical information, medical log and/or an instruction card. The informative guide or information pocket 14 serves to guide the user as to which compartment 5, 6, 7 and 8 carries which accessories so that accidental exposure of accessories can be avoided, by the user opening the wrong compartment. Additionally, the user may easily and safely identify which products to use and where to go in the carrier, without being exposed to the entirety of the products/accessories in the carrier 1, thus relieving the user from undue stress and anxiety. Preferably, the informative guide 14 carries a symbol for each accessory type. The same symbol may then be placed on the corresponding compartment 5, 6, 7 and/or 8. In this way, the user may easily identify on the informative guide what accessory he/she is in need of, and then turn look for the corresponding symbol on the compartment 5, 6, 7, or 8 to retrieve the optimal accessory, i.e. the accessory adapted for the circumstance in question. For this purpose, the information guide may for example have sections for night care, day care, bathroom, going out/leaving house, etc., with a corresponding symbol. For example, the symbol for night care may be a picture of the moon, the symbol for daycare may be a picture of the sun, the symbol for bathroom may be a picture of a toilet or shower, and the symbol for going out/leaving the house may be a picture of a house with a person standing outside the house. Then, the user may identify the same symbol associated with the needed accessory type and find the same symbol on the compartment 5, 6, 7 or 8, for easy accessory type retrieval.

The carrier 1 may also comprise a pocket 12 and/or an informative tag 15 for carrying user identity, user manuals or other documents/papers on the outside of the carrier, as seen in the rear view.

As can be seen from FIG. 1 the first compartment 5 may be folded on to the second compartment 6. The fourth compartment 8 may be folded onto the third compartment 7. As the compartments are folded onto each other, the pockets of each compartment is further ensured to remain closed, especially if covered by flaps as the flaps of each pocket will be pressed towards its pocket by the compartment being folded onto it.

The carrier 1 comprise a lock 10 arranged between the third compartment and the second compartment 6 for engaging a locking strap 9 arranged adjacent the fourth compartment. This ensures that as the fourth compartment is folded on to the third compartment 7 they may be locked in such a partially closed position.

To bring the carrier 1 into a fully closed position a user may thus fold the fourth compartment 8 onto the third compartment 7, lock the lock arrangement 9, 10 (if such is present), fold the first compartment 5 onto the second compartment 6, and possibly also the guide 14, whereby the first and second compartments 5, 6 are folded onto the third and fourth compartments 7,8. The carrier may then be closed by the straps 3 and their locks 4.

As can also be see, the carrier 1 may be arranged with a hook or loop 13 for easy mounting by hanging the carrier when opened. The hook or loop 13 is arranged adjacent the first compartment on a side facing away from the second compartment 6 ensuring that the second and also third and fourth compartments 6, 7, 8 are hanging below the first compartment when being mounted, such as by hanging from a rack or hanger.

FIG. 3 shows a front view of the carrier 1 in a closed informative state according to an embodiment of the teachings herein. As can be seen, the informative guide 14 has been folded away from the third and fourth compartments 7, 8, but is still folded on to the first and second compartments 5, 6. In this closed, but informative, state only information is provided to a user through the guide and possibly through the manuals and/or other medical papers being carried by the information pocket 14. This allows the user to access and/or to consult medical papers, the manuals or other document without having to lay open the compartments carrying the actual accessories.

The first and second compartments 5, 6 and the information pocket 14 are kept in place by a lock 11 (also seen in FIG. 2) engaging a corresponding locking part of the information pocket 14. As for the other locks, the lock 11 may be a hook and loop (such as Velcro™), a magnetic lock or other type of lock exemplified herein.

The third and fourth compartments 7, 8 are kept in place by the lock arrangement 9, 10.

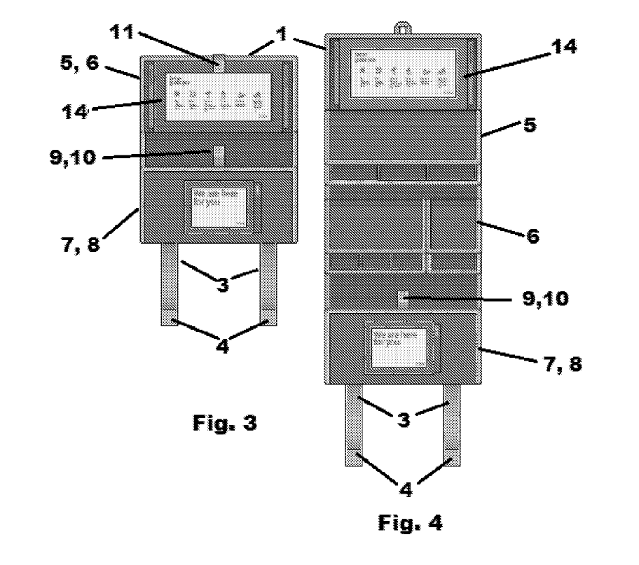

FIG. 4 shows a front view of the carrier 1 in a partially opened state according to an embodiment of the teachings herein. The lock arrangement 11 has been released to allow the first and second compartments 5 and 6 to fold away from each other. The information pocket 14 has also been folded away from the compartments 5, 6.

The first and second compartments 5, 6 are thus made accessible to a user, while the third and fourth compartments 7, 8 are still kept inaccessible and safe guarding the personal integrity of the user. They are kept in place by the lock arrangement 9, 10.

As has been mentioned before, each compartment may comprise one or more pockets (5A, 5B, 5C, 8A, 8B). The pockets may be covered by a foldable flap. In one embodiment, such a flap may cover more than one pocket as is the case for the pockets of the first compartment 5 in this example. A flap may also cover only one pocket, as is the case for the fourth compartment 8. In one embodiment a compartment 5, 6, 7, 8 may comprise two or more subcompartments such as for example for the forth compartment comprising two subcompartments 8A and 8B.

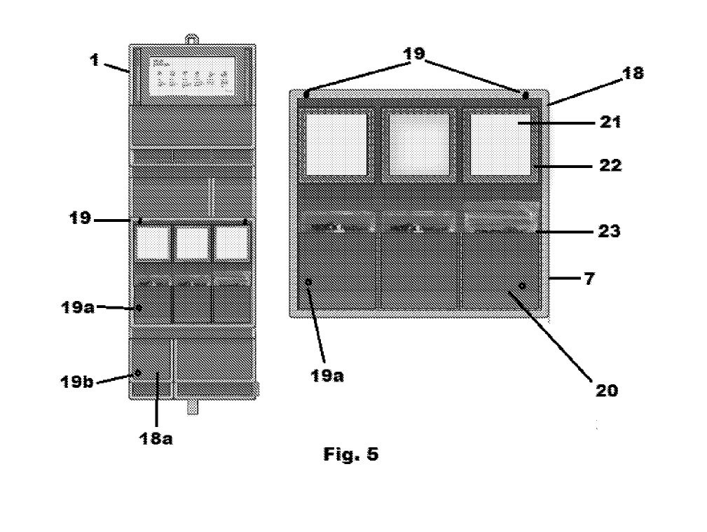

FIG. 5 shows a front view of a carrier 1 having an opened compartment, here exemplified by the third compartment 7, according to an embodiment of the teachings herein and also showing a close up of the opened compartment 7. The compartment 7 comprises at least one pocket 20, in this example three pockets, for carrying or storing the actual accessories 23 to be used by the user. The pockets 23 are covered by a foldable flap 18 which, in this figure, has been folded upwards to reveal its contents. In one embodiment the flap 18 is arranged with at least one frame 22, possibly one for each pocket 20, into which an information card 21 may be slided or printed on.

In one embodiment, the flap 18 is arranged with magnets 19 and corresponding magnets 19a on the pocket 23 for keeping the flap 18 in a closed position.

In one such embodiment, the magnets 19, 19a could be arranged to that each magnet pair 19, 19 on each flap 18 is arranged with an alternating polarity. In other words a magnet pair 19,19a on one flap has an opposite polarity to a magnet pair 19b on an adjacent flap 18a. In the example of FIG. 5 this would mean that the flap 18 for the third compartment 7 would have an opposite polarity to the magnet 19b on the flap 18a for the fourth compartment 8. This allows for a flap 18 to be kept in an open position by the magnet 19 on the flap 18 of one compartment cooperating with the magnet on the flap of an adjacent (or upper) compartment when the flap 18 is folded upwards. In the example of FIG. 5, the flap 18 of the third compartment 7 is kept in an open position by cooperating with magnets (not shown) arranged on the flap of the second compartment. Similarly, should the flap 18a of the fourth compartment 8 be folded upwards (when the third compartment 7 is closed), the magnet 19b on the flap 18a of the fourth compartment 8 would cooperate with the magnet 19 on the flap 18 of the third compartment 7. In one embodiment the magnet on the flap of the first compartment has a positive polarity (seen from a front view when closed), the magnet on the flap of the second compartment has a negative polarity (seen from a front view when closed), the magnet on the flap of the third compartment has a positive polarity (seen from a front view when closed), and the magnet on the flap of the fourth compartment has a negative polarity (seen from a front view when closed).

In such embodiments, the length of a flap 18 equals or exceeds the distance from the carrier structure 1 to the magnet 19 of the compartment above including the thickness of a compartment.

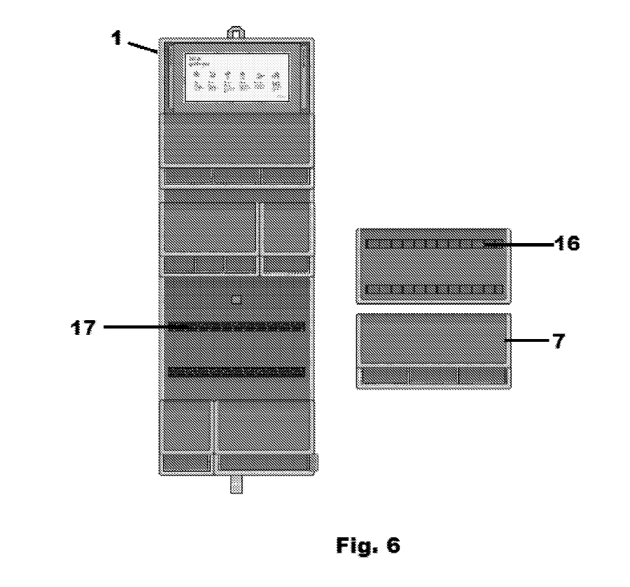

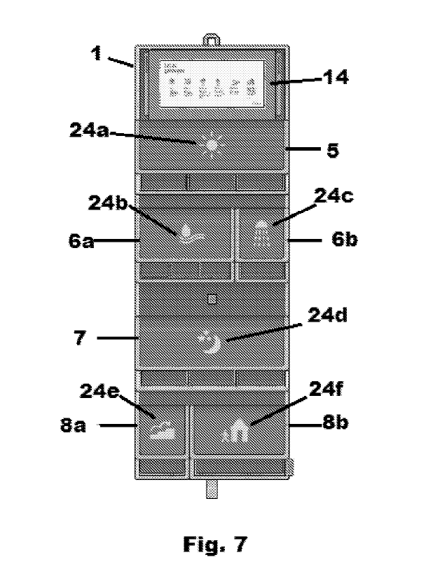

As is shown in FIG. 6 showing a front view of a carrier 1 according to an embodiment of the teachings herein, a compartment 5, 6, 7, 8 may be removably arranged to the carrier 1. FIG. 5 shows the carrier 1 and a front view and a rear view of a removed compartment, here exemplified by the third compartment 7. The removable compartment 7 may be arranged to be fastened to the carrier by a locking arrangement 16, 17 such as a hook and loop locking arrangement (such as Velcro™), push buttons, magnetic locks, zippers or other locking means. A removable compartment has the benefit of allowing the user to only bring the compartment that the user will actually use and does not need to bring the whole carrier 1. This may for example be the compartment comprising the accessories associated with leaving the house.

FIG. 7 shows a front view of a carrier 1 according to an embodiment of the teachings herein. In this example, the flaps of all compartments and also sub compartments 5, 6a, 6b, 7, 8a, 8b have been provided with an informative symbol or labeling such as an icon 24a-f. The icons 24a-f not only presents the user with information on what each compartment 5, 6a, 6b, 7, 8a, 8b carries, but also represent a categorization made possible by the clever arrangement of compartments 5, 6a, 6b, 7, 8a, 8b. The inventors has realized that by categorizing the accessories to be used according to when or what scenario they are to be used in and arranging the accessories of a similar category in the same compartment, possibly in different sub compartments to offer further categorization, it is possible to maintain the personal integrity of a user, while at the same time offer a more convenient manner of accessing an accessory, especially through the use of the compartments that can be maintained inaccessible while some compartments are accessed according to a carrier 1 as disclosed herein.

The categorization also serves to differentiate between such accessories that may be used easily and also may resemble normal products, not necessarily associated with tracheostomy. As the carrier 1 is arranged to be opened in different steps where some compartments remain inaccessible when some compartments are accessible it is thereby possible to maintain the personal integrity of the user by arranging accessories associated with a more intimate use or more closely associated with tracheostomy in a compartment that remains inaccessible in the partially opened state.

In the example of FIG. 7 the first compartment 5 is arranged to carry all accessories associated with (normal) day use. The day use is indicated with a symbol 24a representing a sun. The second compartment 6 is arranged to carry accessories associated with cleaning, and is further divided into two subcompartments 6a, 6b wherein the first 6a is marked by an icon 24b representing waves and a drop indicating skin care use, and the second 6b is marked by an icon 24c representing a shower. The first subcompartment 6a thus carrying accessories associated with skin use and the second subcompartment 6b carrying accessories associated with showering.

The third compartment 7 (which is also one of the two compartments that remain inaccessible in the partially opened state) is arranged with an icon 24d representing a moon, thereby indicating night use and the fourth compartment 8 being divided into two subcompartments a first 8a being marked with an icon 24e indicating advancing, thus carrying more and more advanced accessories, the second subcompartment 8b being marked with an icon 24f representing a person leaving a house indicating outdoor (leaving the house) use. Obviously the contents of the third and fourth compartments 7, 8 would only be accessed inside (most likely ones own house) as they relate to sleeping and also to actions to be taken before going out. As such they are put in compartments that remains inaccessible in the partially opened state and are only accessible when the carrier 1 is fully opened, which would not be a problem in the privacy of ones own home.

A carrier 1 that provides for a categorization of accessories and a manner of maintaining the accessories having been categorized as of a more intimate or advanced type maintained inaccessible even when accessories having been categorized as less intimate are accessed is thus provided, providing for a carrier that maintains the personal integrity of a user while offering a convenient manner of accessing and carrying accessories.

It should be noted that even though the disclosure herein is focused on four compartments 5, 6, 7, 8 and an information pocket 14 any number of pockets is possible, and an information pocket may be present or not. For example, in one embodiment a third and fourth compartment 7, 8 folds onto each other and a first compartment 5 folds on to the folded together third and fourth compartments, thereby providing a similar carrying system having three compartments where the compartments are opened in stages to maintain the personal integrity of the user.

The invention has mainly been described above with reference to a few embodiments. However, as is readily appreciated by a person skilled in the art, other embodiments than the ones disclosed above are equally possible within the scope of the invention, as defined by the appended patent claims.

1. A carrier for carrying tracheostomy accessories, comprising:
a foldable carrier structure including at least two compartments, each compartment of the at least two compartments structured and arranged to carry a type of tracheostomy accessory; and
an informative guide identifying the type of tracheostomy accessory each compartment is structured and arranged to carry
wherein, when the carrier structure is folded in a closed state, the at least two compartments are folded onto one another such that the at least two compartments are inaccessible.

2. The carrier according to claim 1, wherein each compartment includes a symbol indicating an associated tracheostomy accessory category, the type of tracheostomy accessory each compartment is structured and arranged to carry falling within the associated tracheostomy accessory category.

3. The carrier according to claim 1, wherein:
at least two compartments includes four compartments including a first compartment, a second compartment, a third compartment, and a fourth compartment, the carrier structure foldable such that the third compartment is folded onto the fourth compartment; and
the carrier structure further includes a locking arrangement configured to keep the third compartment folded on the fourth compartment when the carrier structure is folded in a partially opened state.

4. The carrier according to claim 3, wherein:
the carrier structure is foldable such that the first compartment is folded onto the second compartment; and
when the carrier structure is folded in the closed state, the first compartment folded on the second compartment is folded onto the third compartment folded on the fourth compartments.

5. The carrier according to claim 4, wherein:
the carrier structure further includes another locking assembly configured to keep the first compartment folded on the second compartment when the carrier structure is folded in an information state; and
when the carrier structure is folded in the information state, the first compartment folded on the second compartments is not folded onto the third compartment folded on the fourth compartment.

6. The carrier according to claim 1, wherein the at least two compartments are respectively configured as a pocket structured and arranged to carry at least a portion of a tracheostomy accessory and include a cover flap configured to cover the pocket, wherein the cover flap of a lower compartment of the at least two compartments includes a magnet configured to engage an oppositely polarized magnet of the cover flap of an upper compartment of the at least two compartments when the cover flap of the lower compartment is in an open position such that the cover flap of the lower compartment is held in the open position.

7. The carrier according to claim 1, wherein at least one of the at least two compartments includes at least two sub-compartments.

8. The carrier according to claim 1, wherein at least one of the at least two compartments is removably arranged on the carrier carrier structure.

9. The carrier according to claim 1, further comprising at least one locking mechanism configured to keep the carrier structure folded in the closed state.

10. The carrier according to claim 9, wherein the at least one locking mechanism is one of a strap, a magnetic snaplock, a button lock, a snap lock, and a hook and loop arrangement.

11. The carrier according to claim 1, wherein each compartment is openable and closable via one of a flap, a zipper, and a zip lock.

12. The carrier according to claim 1, wherein the carrier structure further includes at least one document compartment configured to carry at least one of a personal identification, a user manual, and a medical record history.

13. The carrier according to claim 12, wherein the at least one document compartment is arranged on the carrier structure such that the at least one document compartment is disposed on an exterior of the carrier structure at least when the carrier structure is folded in the closed position.

14. The carrier according to claim 1, wherein the carrier support is hangable via a mounting mechanism coupled to the carrier support.

15. A carrier for carrying tracheostomy accessories, comprising:
a foldable carrier structure including a plurality of compartments defined by a first compartment, a second compartment, a third compartment, and a forth compartment, each compartment of the plurality of compartments structured and arranged to carry a type of tracheostomy accessory; and
an informative guide arranged on the carrier structure identifying the type of tracheostomy accessory each compartment is structured and arranged to carry;
wherein the plurality of compartments are arranged on the carrier structure such that when the carrier structure is folded into a closed state the plurality of compartments are folded onto one another and are inaccessible.

16. The carrier according to claim 15, wherein:
the first compartment and the second compartment are arranged on an upper portion of the carrier structure such that the first compartment and the second compartment abut one another and are inaccessible when the upper portion of the carrier structure is folded into an upper-closed state;
the third compartment and the forth compartment are arranged on a lower portion of the carrier structure such that the third compartment and the forth compartment abut one another and are inaccessible when the lower portion of the carrier structure is folded into a lower-closed state; and
the upper portion in the upper-closed state and the lower portion in the lower-closed state are folded onto one another when the carrier structure is folded in the closed state.

17. The carrier according to claim 16, wherein the informative guide is arranged on the carrier structure such that i) the informative guide is visible when the carrier structure is folded in at least one of the upper-closed state and the lower-closed state and ii) the informative guide is not visible when the carrier structure is folded in the closed state.

18. The carrier according to claim 16, further comprising:
a first locking arrangement configured to secure the carrier structure in the upper-closed state; and
a second locking arrangement configured to secure the carrier structure in the lower-closed state.

19. The carrier according to claim 15, wherein each compartment includes a symbol indicating an associated tracheostomy accessory category, the type of tracheostomy accessory each compartment is structured and arranged to carry falling within the associated tracheostomy accessory category.

20. A carrier for carrying tracheostomy accessories, comprising:
a foldable carrier structure;
a plurality of compartments defined by a first compartment, a second compartment, a third compartment, and a forth compartment arranged on one after another on the foldable carrier, each compartment of the plurality of compartments structured and arranged to retain a tracheostomy accessory; and
an informative guide arranged on the carrier structure identifying the type of tracheostomy accessory each compartment is structured and arranged to retain;
wherein an upper portion of the carrier structure including the first compartment is foldable inwardly onto the second compartment to define an upper-folded portion, and a lower portion of the carrier structure including the forth compartment is foldable inwardly onto the third compartment to define a lower-folded portion; and
wherein, when the carrier structure is folded into a closed state, the upper-folded portion is folded onto the lower-folded portion such that i) the first compartment is disposed between the second compartment and the fourth compartment, ii) the forth compartment is disposed between the first compartment and the third compartment, and ii) any tracheostomy accessory retained by the plurality of compartments are inaccessible.
